(19) United States
(12) Patent Application Publication
(10) Pub. No.: US 2018/0246194 A1
(43) Pub. Date: Aug. 30, 2018

(54) ULTRASONIC IMAGE PROCESSING APPARATUS AND ULTRASONIC IMAGE PROCESSING METHOD

(71) Applicant: Seiko Epson Corporation, Tokyo (JP)

(72) Inventor: Kenji MURAKAMI, Shiojiri (JP)

(21) Appl. No.: 15/865,620

(22) Filed: Jan. 9, 2018

(30) Foreign Application Priority Data
Feb. 27, 2017 (JP) 2017-034466

(51) Int. Cl. G01S 7/52; G01S 7/531; A61B 8/00

(52) U.S. Cl. CPC G01S 7/52085; A61B 8/4477; G01S 7/52079; G01S 7/531

(57) ABSTRACT

An ultrasonic image processing apparatus includes pixels arranged in a first axis direction corresponding to a scanning direction of an ultrasonic wave transmitted to an object and a second axis direction corresponding to a distance direction in which the ultrasonic wave propagates. Each of the pixels has coordinates based on a reflection position of the ultrasonic wave and a pixel value based on a strength of a reflected wave of the ultrasonic wave. The ultrasonic image processing apparatus includes: a speckle pattern reduction processor setting a size of a filter according to a coordinate on the second axis of a pixel of interest included in the ultrasonic image and performing filter processing using the filter to reduce a speckle pattern in the pixel of interest; and an edge information calculator calculating edge information for the pixel of interest in which the speckle pattern has been reduced.

### BACKGROUND

### 1. Technical Field

The present invention relates to an ultrasonic image processing apparatus and an ultrasonic image processing method.

### 2. Related Art

In an ultrasonic image, not only information regarding the tissue of a subject but also speckles generated due to various kinds of noise or the interference phenomenon of ultrasonic reception signals are present. For this reason, in the case of performing blurring processing in order to remove various kinds of noise or speckles, there is a problem that the boundary position or the shape of the tissue of the subject becomes unclear. In order to solve this problem, JP-A-2011-125757 discloses an ultrasonic image data processing apparatus including: a unit that sets a plurality of line segments having different directions passing through a pixel of interest on ultrasonic image data, which is obtained by transmission and reception of ultrasonic waves, and that calculates a variance value for each line segment based on a plurality of pixel values of a pixel column on the line segment; a first specification unit that specifies a first direction corresponding to the normal direction of the boundary based on a plurality of calculated variance values calculated for the plurality of line segments; a unit that specifies a second direction along the boundary as a direction that is perpendicular to the first direction and passes through the pixel of interest; and a smoothing operation unit that calculates a smoothing pixel value of the pixel of interest based on a plurality of pixel values of pixel columns arranged in the second direction. According to the ultrasonic image data processing apparatus, by using the variance values of line segments in various directions from the pixel of interest, a direction perpendicular to a direction in which the variance value increases is set as an edge direction. Therefore, it is possible to reduce the blurring of the boundary between tissues.

However, since the ultrasonic image data processing apparatus disclosed in JP-A-2011-125757 calculates the variance value from only a plurality of pixel values of the pixel column on each line segment, the calculated variance value is greatly affected by noise on the line segment or the like. For this reason, an edge is extracted for a flat portion, such as a speckle noise region. Therefore, the ultrasonic image data processing apparatus disclosed in JP-A-2011-125757 has a problem that noise is also emphasized in a case where edge emphasis processing is performed based on the specified edge direction in order to clarify the boundary position or the shape of the tissue of the subject.

### SUMMARY

An advantage of some aspects of the invention is to provide an ultrasonic image processing apparatus and an ultrasonic image processing method capable of accurately calculating the edge information of a pixel of interest for an ultrasonic image.

The invention can be implemented as the following forms or application examples.

### Application Example 1

An ultrasonic image processing apparatus according to this application example is an ultrasonic image processing apparatus for processing an ultrasonic image including a plurality of pixels arranged in a direction of a first axis corresponding to a scanning direction of an ultrasonic wave transmitted to an object and a direction of a second axis corresponding to a distance direction in which the ultrasonic wave propagates. Each of the plurality of pixels has coordinates based on a reflection position of the ultrasonic wave and a pixel value based on a strength of a reflected wave of the ultrasonic wave. The ultrasonic image processing apparatus includes: a speckle pattern reduction processing section that sets a size of a filter according to a coordinate on the second axis of a pixel of interest included in the ultrasonic image and performs filter processing using the filter to reduce a speckle pattern in the pixel of interest; and an edge information calculation section that calculates edge information for the pixel of interest in which the speckle pattern has been reduced.

Since nonlinear components are generated according to the propagation of the ultrasonic wave, the waveform of the ultrasonic wave becomes dull and the ultrasonic wave attenuates (has a lower frequency). As a result, in the ultrasonic image, the size of the speckle pattern changes according to the coordinate on the second axis. In the ultrasonic image processing apparatus according to this application example, filter processing is performed on the pixel of interest by setting the size of the filter according to the coordinate on the second axis corresponding to the distance direction in which the ultrasonic wave propagates. Accordingly, it is possible to effectively reduce the speckle pattern by using a filter having an appropriate size corresponding to the size of the speckle pattern and to suppress blurring of the edge (boundary between tissues inside the object) due to smoothing as much as possible. Therefore, according to the ultrasonic image processing apparatus according to this application example, it is possible to accurately calculate edge information (information regarding edges) for the pixel of interest in which the speckle pattern has been effectively reduced.

### Application Example 2

In the ultrasonic image processing apparatus according to the application example, the speckle pattern reduction processing section may set the size of the filter in the second axis direction according to the coordinate of the pixel of interest on the second axis.

According to the ultrasonic image processing apparatus according to this application example, it is possible to effectively reduce the speckle pattern using a filter, which has an appropriate size in the second axis direction corresponding to the size of the speckle pattern, for the pixel of interest. In addition, it is possible to suppress blurring of edges due to smoothing as much as possible. Therefore, according to the ultrasonic image processing apparatus according to the application example, it is possible to accurately calculate edge information for the pixel of interest in which the speckle pattern has been effectively reduced.

### Application Example 3

In the ultrasonic image processing apparatus according to the application example, the speckle pattern reduction processing section may set the size of the filter in the first axis direction according to the coordinate of the pixel of interest on the second axis.

According to the ultrasonic image processing apparatus according to this application example, it is possible to effectively reduce the speckle pattern using a filter, which has an appropriate size in the first axis direction corresponding to the size of the speckle pattern, for the pixel of interest. In addition, it is possible to suppress blurring of edges due to smoothing as much as possible. Therefore, according to the ultrasonic image processing apparatus according to the application example, it is possible to accurately calculate edge information for the pixel of interest in which the speckle pattern has been effectively reduced.

### Application Example 4

In the ultrasonic image processing apparatus according to the application example, the speckle pattern reduction processing section may set the size of the filter for a first pixel of interest to be equal to or greater than the size of the filter for a second pixel of interest whose coordinate on the second axis is smaller than that of the first pixel of interest.

According to the ultrasonic image processing apparatus according to this application example, filter processing is performed on the pixel of interest using the pixel values of a larger number of pixels by setting the size of the filter so that the size of the filter increases as the coordinate on the second axis increases. Therefore, it is possible to effectively reduce the speckle pattern, which increases as the coordinate on the second axis increases, and to suppress blurring of edges due to smoothing as much as possible. Therefore, according to the ultrasonic image processing apparatus according to the application example, it is possible to accurately calculate edge information for the pixel of interest in which the speckle pattern has been effectively reduced.

### Application Example 5

The ultrasonic image processing apparatus according to the application example may further include: an ultrasonic image generation section that generates the ultrasonic image based on a reflected wave of the ultrasonic wave transmitted to the object.

According to the ultrasonic image processing apparatus according to this application example, in the ultrasonic image generated based on the reflected wave of the ultrasonic wave transmitted to the object, it is possible to effectively reduce the speckle pattern for the pixel of interest and to suppress blurring of edges due to smoothing as much as possible. As a result, it is possible to accurately calculate edge information for the pixel of interest.

### Application Example 6

The ultrasonic image processing apparatus according to the application example may further include: an image correction section that corrects the ultrasonic image based on the edge information.

According to the ultrasonic image processing apparatus according to this application example, by correcting the ultrasonic image based on the edge information calculated accurately for the pixel of interest, it is possible to generate a clearer ultrasonic image in which the sharpness of a region including an edge (edge region) is emphasized.

### Application Example 7

An ultrasonic image processing method according to this application example is an ultrasonic image processing method for processing an ultrasonic image including a plurality of pixels arranged in a direction of a first axis corresponding to a scanning direction of an ultrasonic wave transmitted to an object and a direction of a second axis corresponding to a distance direction in which the ultrasonic wave propagates. Each of the plurality of pixels has coordinates based on a reflection position of the ultrasonic wave and a pixel value based on a strength of a reflected wave of the ultrasonic wave. The ultrasonic image processing method includes: setting a size of a filter according to a coordinate on the second axis of a pixel of interest included in the ultrasonic image; performing filter processing using the filter to reduce a speckle pattern in the pixel of interest; and calculating edge information for the pixel of interest in which the speckle pattern has been reduced.

Since nonlinear components are generated according to the propagation of the ultrasonic wave, the waveform of the ultrasonic wave becomes dull and the ultrasonic wave attenuates (has a lower frequency). As a result, in the ultrasonic image, the size of the speckle pattern changes according to the coordinate on the second axis. In the ultrasonic image processing method according to this application example, filter processing is performed on the pixel of interest by setting the size of the filter according to the coordinate on the second axis corresponding to the distance direction in which the ultrasonic wave propagates. Accordingly, it is possible to effectively reduce the speckle pattern by using a filter having an appropriate size corresponding to the size of the speckle pattern and to suppress blurring of the edge due to smoothing as much as possible. Therefore, according to the ultrasonic image processing method according to the application example, it is possible to accurately calculate edge information for the pixel of interest in which the speckle pattern has been effectively reduced.

### BRIEF DESCRIPTION OF THE DRAWINGS

The invention will be described with reference to the accompanying drawings, wherein like numbers reference like elements.

FIG. 1 is a diagram showing an example of the appearance of an ultrasonic image apparatus according to the present embodiment.

FIG. 2 is a schematic diagram of the internal configuration of an ultrasonic transducer device.

FIG. 3 is a diagram showing how ultrasonic waves are sequentially transmitted in a linear scan.

FIG. 4 is a diagram showing how ultrasonic waves are sequentially transmitted in a sector scan.

FIG. 5 is a diagram showing the ultrasonic wave UW-k shown in FIG. 4 in more detail.

FIG. 6 is a diagram showing an example of the configuration of an ultrasonic image processing apparatus.

FIG. 7 is a block diagram showing an example of the functional configuration of a processing unit.

FIG. 8 is a diagram showing an example of the sound pressure waveform of ultrasonic waves.

FIG. 9 is a diagram showing an example of an ultrasonic image generated by an ultrasonic image generation section.

FIG. 10 is a flowchart showing the procedure of ultrasonic image processing of an ultrasonic image processing section.

FIG. 11 is a diagram showing an example of the relationship between each region of an ultrasonic image and a filter size to be applied.

FIG. 12 is a flowchart showing an example of the procedure of speckle pattern reduction processing.

FIG. 13 is a diagram showing an example of a filter used for calculation of an edge strength in a scanning direction (x direction).

FIG. 14 is a diagram showing an example of a filter used for calculation of an edge strength in a distance direction (z direction).

FIG. 15 is a diagram showing the relationship among the edge strength and the edge direction, the edge strength in the scanning direction (x direction), and the edge strength in the distance direction (z direction).

FIG. 16 is a flowchart showing an example of the procedure of edge information calculation processing.

FIG. 17 is a flowchart showing an example of the procedure of smoothing processing.

FIG. 18 is a diagram showing an example of a filter used for edge sharpening processing.

FIG. 19 is a flowchart showing an example of the procedure of edge sharpening processing.

FIG. 20 is a flowchart showing an example of the procedure of image combining processing.

### DESCRIPTION OF EXEMPLARY EMBODIMENTS

Hereinafter, preferred embodiments of the invention will be described in detail with reference to the accompanying diagrams. The embodiments described below are not intended to limit the contents of the invention defined by the appended claims. In addition, all of the configurations described below are not necessarily essential components of the invention.

### 1. Configuration of Ultrasonic Image Apparatus

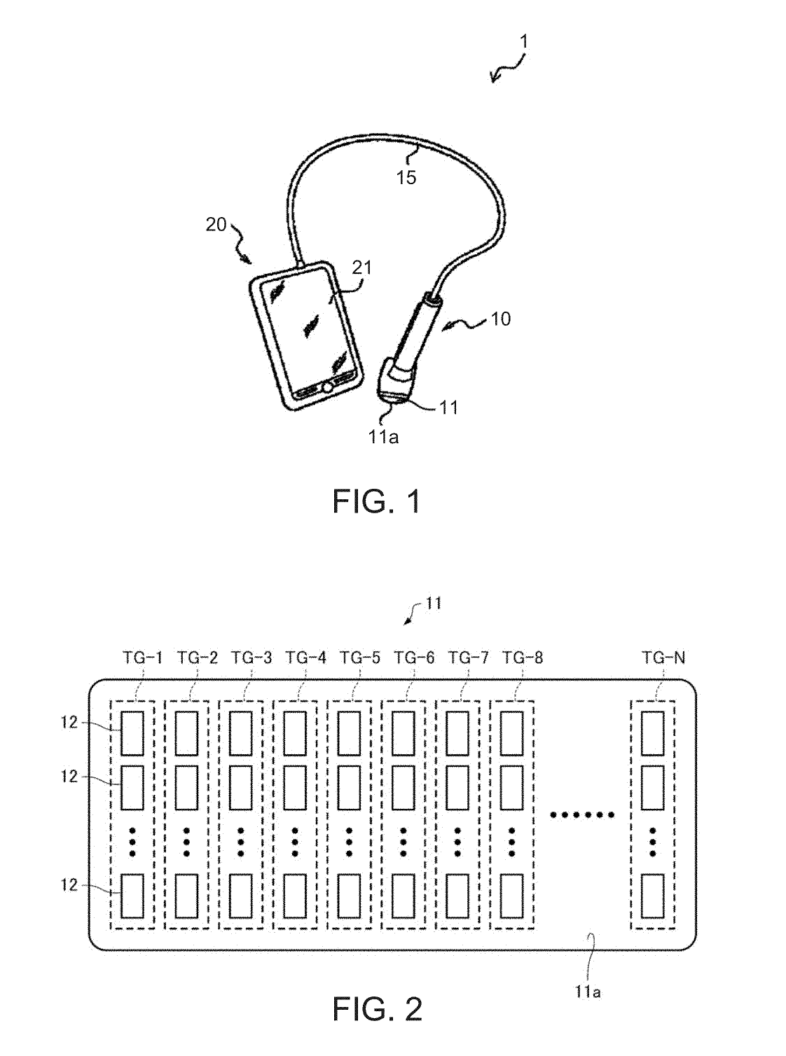

FIG. 1 is a diagram showing an example of the appearance of an ultrasonic image apparatus according to the present embodiment. An ultrasonic image apparatus 1 according to the present embodiment is configured to include an ultrasonic probe 10 and an ultrasonic image processing apparatus 20. The ultrasonic probe 10 and the ultrasonic image processing apparatus 20 are connected to each other by a cable 15. The ultrasonic image processing apparatus 20 may be a portable type apparatus, or may be a fixed type (stationary type) apparatus. The ultrasonic probe 10 may be built into the ultrasonic image processing apparatus 20.

The ultrasonic probe 10 has an ultrasonic transducer device 11. The ultrasonic transducer device 11 transmits an ultrasonic wave to an object and receives a reflected ultrasonic wave (reflected wave) on a predetermined surface (transmission and reception surface 11a) while scanning the object.

FIG. 2 is a schematic diagram of an internal configuration in a case where the ultrasonic transducer device 11 is seen from the bottom surface (measurement surface). As shown in FIG. 2, the ultrasonic transducer device 11 has a plurality of ultrasonic transducer elements 12 arranged in a matrix. More specifically, the ultrasonic transducer device 11 has N ultrasonic transducer element groups TG-1 to TG-N arranged side by side along the scanning direction, and each of the ultrasonic transducer element groups TG-1 to TG-N has a plurality of ultrasonic transducer elements 12 arranged along a direction (slice direction) perpendicular to the scanning direction. The ultrasonic transducer element 12 can be formed using a piezoelectric element formed of a material, such as lead zirconate titanate (PZT), lead titanate (PbTiO₃), lead zirconate (PbZrO₃), and lead lanthanum titanate ((Pb, La)TiO₃). For example, the ultrasonic transducer element 12 has a monomorph (unimorph) structure in which a thin piezoelectric element and a metal plate (vibration film) are bonded to each other.

Each of the ultrasonic transducer element groups TG-1 to TG-N forms one channel driven in the transmission of ultrasonic waves. Therefore, hereinafter, the ultrasonic transducer element groups TG-1 to TG-N will be referred to as “channel 1” to “channel N”, respectively.

As a method of scanning the object using the ultrasonic transducer device 11 having such a structure, for example, a linear scan or a sector scan is possible.

In the linear scan, ultrasonic waves are transmitted from a plurality of channels (for example, eight channels) among the N channels while shifting the channels. In the sector scan, ultrasonic waves are transmitted from a predetermined channel (for example, all N channels) while changing the direction (angle).

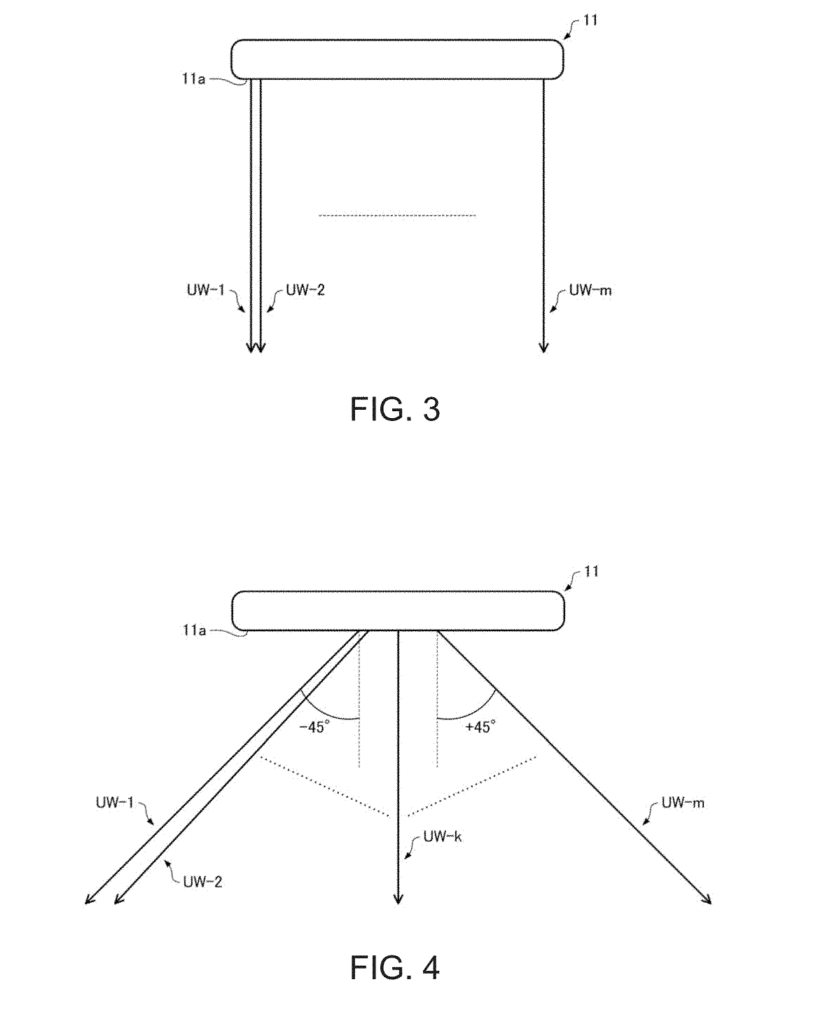

FIG. 3 is a diagram showing how ultrasonic waves are sequentially transmitted in the linear scan, and FIG. 4 is a view showing how ultrasonic waves are sequentially transmitted in the sector scan. FIGS. 3 and 4 are diagrams of the ultrasonic transducer device 11 as viewed from the side surface. The ultrasonic wave transmitted from each of the ultrasonic transducer elements 12 is a spherical wave, and a plurality of ultrasonic waves transmitted from a plurality of channels interfere with each other to be combined. As a result, composite m ultrasonic waves UW-1 to UW-m are sequentially transmitted.

As shown in FIG. 3, in the linear scan, the ultrasonic transducer device 11 sequentially transmits the ultrasonic waves UW-1 to UW-m while changing the channel. For example, the ultrasonic wave UW-1 is a composite wave of a plurality of ultrasonic waves transmitted from the channels 1 to 8, and the ultrasonic wave UW-2 is a composite wave of a plurality of ultrasonic waves transmitted from the channels 2 to 9 after a predetermined time from the transmission of the ultrasonic wave UW-1.

As shown in FIG. 4, in the sector scan, the ultrasonic transducer device 11 fixes the channel, and sequentially transmits the ultrasonic waves UW-1 to UW-m while changing the scanning angle. For example, the ultrasonic wave UW-1 is a composite wave of a plurality of ultrasonic waves transmitted from a plurality of channels with a time difference so that the ultrasonic wave from the channel farther from the channel 1 is transmitted earlier, and the scanning angle is −45°. The ultrasonic wave UW-k (k=N/2) is a composite wave of a plurality of ultrasonic waves transmitted from a plurality of channels with a time difference so that the ultrasonic wave from the channel farther from the channel k is transmitted earlier, and the scanning angle is 0°. The ultrasonic wave UW-m is a composite wave of a plurality of ultrasonic waves transmitted from a plurality of channels with a time difference so that the ultrasonic wave from the channel farther from the channel N is transmitted earlier, and the scanning angle is +45°.

The ultrasonic transducer device 11 may sequentially transmit the ultrasonic waves UW-1 to UW-m while changing both the channel and the scanning angle.

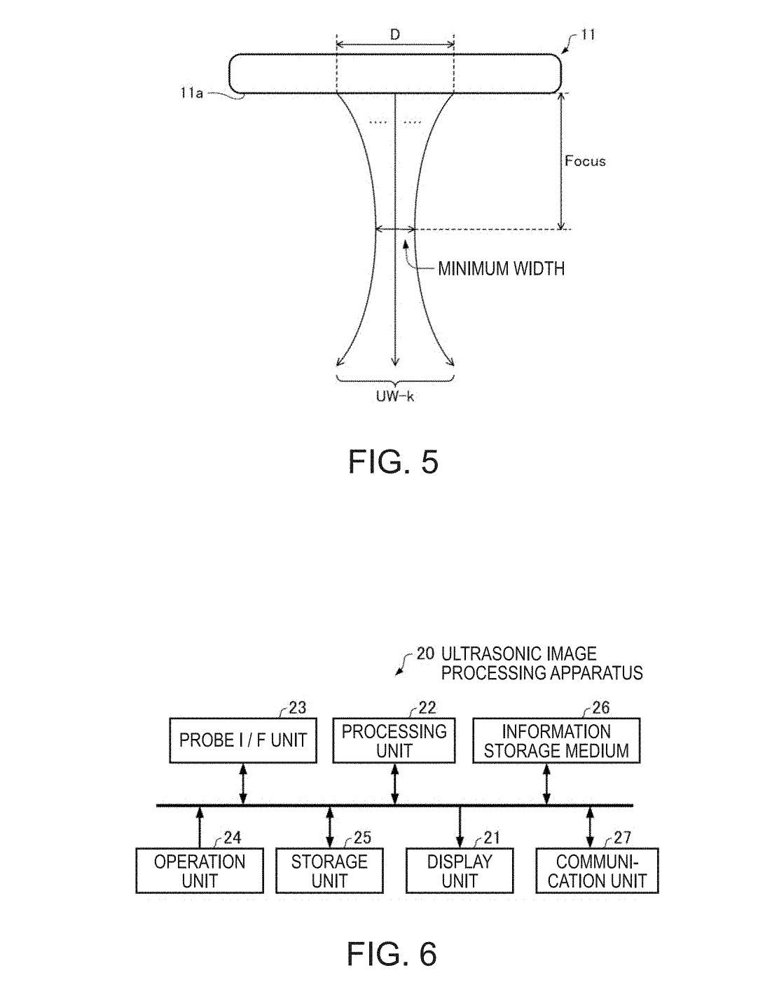

In practice, each of the ultrasonic waves UW-1 to UW-m has a width (beam width) since each of the ultrasonic waves UW-1 to UW-m is a composite wave of a plurality of ultrasonic waves, and the beam width of each of the ultrasonic waves UW-1 to UW-m changes according to the propagation distance. FIG. 5 is a diagram showing the ultrasonic wave UW-k shown in FIG. 4 in more detail. As shown in FIG. 5, the beam width of the ultrasonic wave UW-k is a width corresponding to a plurality of channels to be driven at the transmission start position (generation position), and narrows and then increases as the propagation distance increases. Hereinafter, a position at which the beam width is minimized (focal length from the transmission and reception surface 11a of the ultrasonic transducer device 11) will be referred to as a “transmission focus position”. The transmission focus position Focus can be adjusted by adjusting the timing at which ultrasonic waves are transmitted from a plurality of channels to be driven. Hereinafter, the beam width at the transmission start position (generation position) of ultrasonic waves will be referred to as a “transmission aperture diameter”. A transmission aperture diameter D is determined by the number of channels for transmitting ultrasonic waves, the width of the ultrasonic transducer element 12, and the like.

The ultrasonic waves UW-1 to UW-m are reflected inside the object, the reflected waves are incident on the transmission and reception surface 11a of the ultrasonic probe 10 and are converted into electrical signals by the ultrasonic transducer elements 12.

The ultrasonic image processing apparatus 20 receives the electrical signal from the ultrasonic transducer device 11, calculates the reflection position (distance) of the ultrasonic wave from the information of the ultrasonic wave transmission channel or the scanning angle, the strength of the received signal, and the like, and generates an ultrasonic image in which the horizontal axis is a scanning direction (also referred to as an “azimuth direction”) and the vertical axis is a distance direction (also referred to as an “depth direction”). Then, the ultrasonic image processing apparatus 20 performs image correction processing or image adjustment processing on the generated ultrasonic image, and displays the ultrasonic image on a display unit 21.

### 2. Configuration of Ultrasonic Image Processing Apparatus

FIG. 6 is a diagram showing an example of the configuration of the ultrasonic image processing apparatus 20. As shown in FIG. 6, the ultrasonic image processing apparatus 20 is configured to include the display unit 21, a processing unit 22, a probe interface (I/F) unit 23, an operation unit 24, a storage unit 25, an information storage medium 26, and a communication unit 27. The ultrasonic image processing apparatus 20 may be, for example, a personal computer.

The processing unit 22 performs various kinds of processing based on programs or data stored in the information storage medium 26, various kinds of setting information stored in the storage unit 25, signals input from the operation unit 24, and the like. In the present embodiment, the processing unit 22 performs processing for transmitting a driving signal (pulse signal) to the ultrasonic probe 10, processing for receiving the signal from the ultrasonic probe 10 and generating an ultrasonic image, image processing on the generated ultrasonic image, and the like.

The probe interface unit 23 is an interface unit for establishing transmission and reception of signals between the processing unit 22 and the ultrasonic probe 10.

The operation unit 24 is for inputting the operation of the user or the like as data, and its function can be realized by hardware, such as a keyboard or a mouse.

The storage unit 25 serves as a work area of the processing unit 22, and its function can be realized by hardware, such as a RAM. Various kinds of setting information for controlling the operation of the processing unit 22 and the like are stored in the storage unit 25.

The information storage medium 26 (computer-readable medium) stores programs, data, and the like, and its function can be realized by hardware, such as an optical disc (CD, DVD, and the like), a magneto-optical disc (MO), a magnetic disk, a hard disk, a magnetic tape, or a memory (ROM).

The display unit 21 is for outputting ultrasonic images and the like generated and processed by the processing unit 22, and its function can be realized by hardware, such as a CRT display, a liquid crystal display (LCD), an organic EL display (OELD), a plasma display panel (PDP), and a touch panel type display.

The communication unit 27 performs various kinds of control for communicating with an external device (for example, a server device or a terminal device).

Various programs executed by the processing unit 22 may be distributed from the information storage medium of the server device or the like to the information storage medium 26 (storage unit 25) through the network and the communication unit 27.

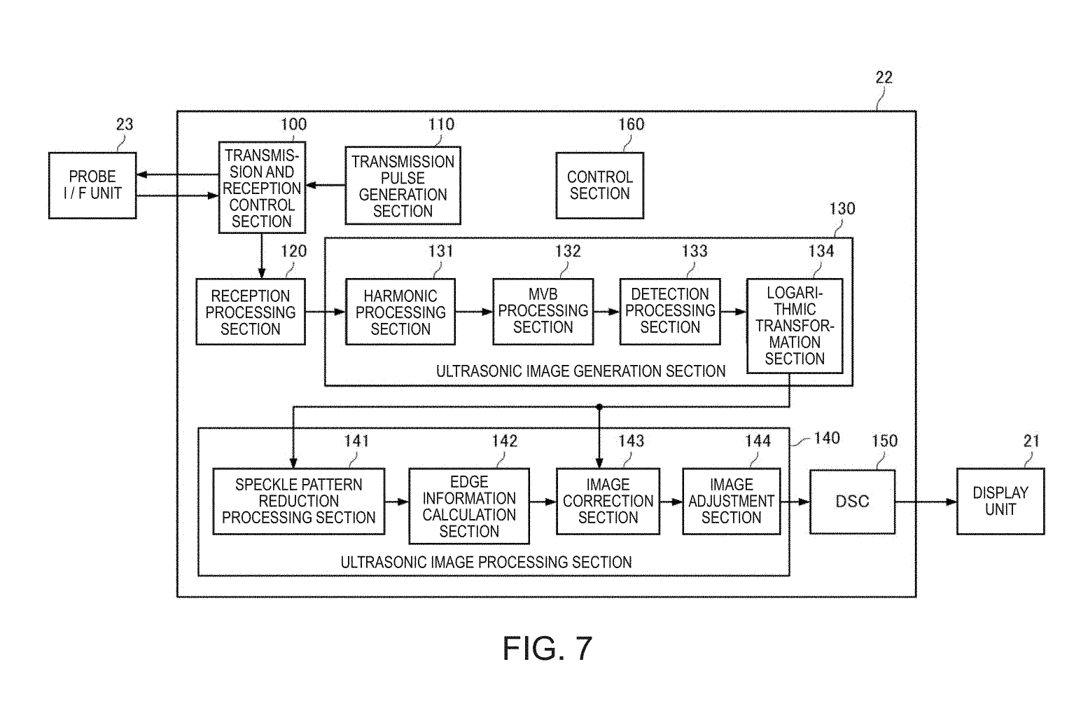

FIG. 7 is a block diagram showing an example of the functional configuration of the processing unit 22. In the example shown in FIG. 7, the processing unit 22 is configured to include a transmission and reception control section 100, a transmission pulse generation section 110, a reception processing section 120, an ultrasonic image generation section 130, an ultrasonic image processing section 140, a digital scan converter (DSC) 150, and a control section 160. The configuration of at least a part of the processing unit 22 shown in FIG. 7 may be provided in the ultrasonic probe 10.

The transmission pulse generation section 110 generates a pulse signal for driving the ultrasonic transducer device 11 included in the ultrasonic probe 10.

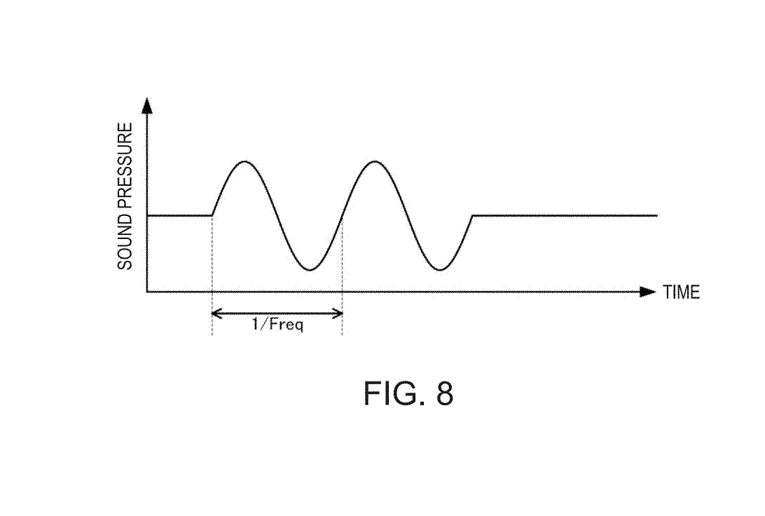

The transmission and reception control section 100 selects a channel for generating ultrasonic waves, and transmits a pulse signal generated by the transmission pulse generation section 110 to the selected channel through the probe interface unit 23. Then, each ultrasonic transducer element 12 included in the selected channel generates an ultrasonic wave having a transmission frequency and a transmission wave number corresponding to the pulse signal. FIG. 8 shows the sound pressure waveform of an ultrasonic wave having a transmission frequency Freq (period: 1/Freq) and a transmission wave number of 2. The transmission and reception control section 100 controls the transmission focus position (focal length) or the scanning angle of ultrasonic waves by delaying the transmission timing of the pulse signal to each channel by a delay time set for each channel so that the transmission timing between the channels is shifted.

After transmitting the pulse signal, the transmission and reception control section 100 receives a reception signal (electrical signal corresponding to the reflected wave of the ultrasonic wave) from each channel of the ultrasonic transducer device 11 through the probe interface unit 23, and outputs each reception signal to the reception processing section 120.

The reception processing section 120 converts the reception signal (analog signal) for each channel into a digital signal, reduces noise by performing filter processing using a band pass filter or the like, and stores the digital signal after the noise reduction in the storage unit 25 (refer to FIG. 6).

The ultrasonic image generation section 130 is configured to include a harmonic processing section 131, a minimum variance beamforming (MVB) processing section 132, a detection processing section 133, and a logarithmic transformation section 134.

The harmonic processing section 131 acquires the reception signal stored in the storage unit 25, and extracts a signal of a harmonic component (also referred to as a harmonic component) for each channel. For example, the harmonic processing section 131 extracts only the second harmonic.

The MVB processing section 132 performs MVB processing, which is adaptive beamforming with direction constraint, based on the signal of the harmonic component for each channel extracted by the harmonic processing section 131. Specifically, the MVB processing section 132 delays the harmonic component signals of the respective channels, converts the harmonic component signals into signals having the same phase of the respective channels, and weights and adds the signals of the respective channels. Here, the weight of each channel is dynamically changed so that the variance value of the result of weighted addition is minimized. That is, the MVB processing is processing for dynamically changing the sensitivity characteristic by changing the weight of each channel according to the reception signal, so that no sensitivity regarding unnecessary waves is obtained.

The detection processing section 133 performs absolute value (rectification) processing on the signal for each channel subjected to the MVB processing by the MVB processing section 132, and then performs filter processing using a low pass filter to extract the signal strength.

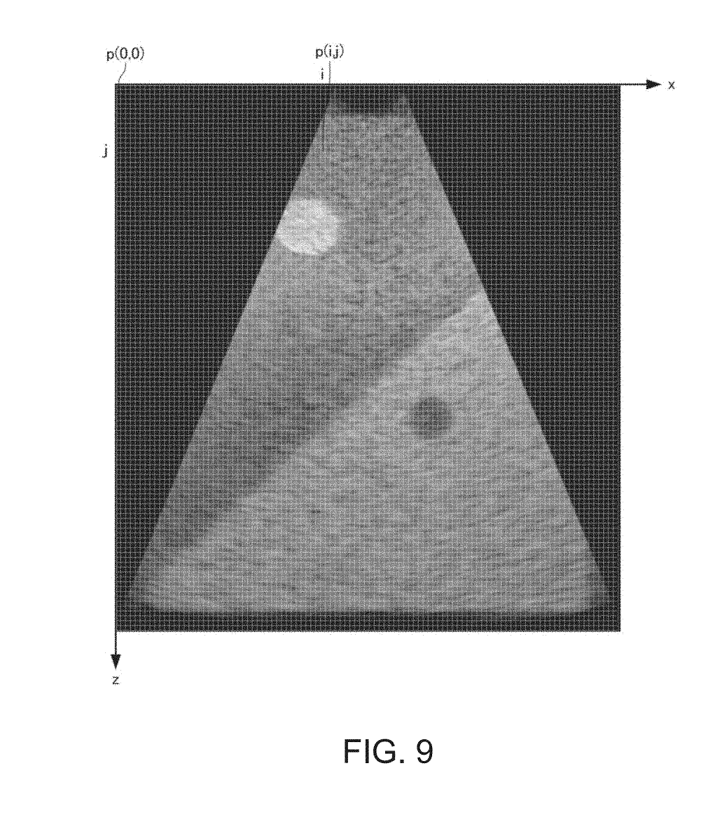

The logarithmic transformation section 134 performs Log compression on the signal strength for each channel extracted by the detection processing section 133, and converts the signal into a signal with a small difference between the maximum value and the minimum value of the signal strength. Then, the signal (signal strength of the reception signal) output from the logarithmic transformation section 134 is stored in the storage unit 25 (refer to FIG. 6) so as to match the position coordinates of the object in the scanning direction (azimuth direction) and the position coordinates of the object in the distance direction (depth direction) inside the object. Based on the data stored in the storage unit 25, it is possible to draw an ultrasonic image including a plurality of pixels arranged in a direction of the horizontal axis (an example of the “first axis”) corresponding to the scanning direction (azimuth direction) of the ultrasonic wave transmitted to the object and a direction of the vertical axis (an example of the “second axis”) corresponding to the distance direction (depth direction) in which the ultrasonic wave propagates. Each of the plurality of pixels included in the ultrasonic image has coordinates based on the reflection position of the ultrasonic wave and a pixel value based on the strength of the reflected wave of the ultrasonic wave. That is, it can be said that the ultrasonic image generation section 130 generates an ultrasonic image based on the reflected wave of the ultrasonic wave transmitted to the object. FIG. 9 is a diagram showing an example of the ultrasonic image generated by the ultrasonic image generation section 130. In FIG. 9, the horizontal axis (x axis) corresponds to the scanning direction (azimuth direction), and the vertical axis (z axis) corresponds to the distance direction (depth direction). For example, a pixel value P_{i, j} of a pixel p(i, j) whose x coordinate is i and z coordinate is j is an integral values in the range of 0 to 255 corresponding to the strength of the reflected wave reflected at a position corresponding to the coordinates (i, j) inside the object. The pixel value is, for example, a brightness value. In FIG. 9, a pixel having a larger pixel value (strength of the reflected wave) is drawn in white.

The ultrasonic image processing section 140 performs predetermined image processing on the ultrasonic image generated by the ultrasonic image generation section 130. Specifically, the ultrasonic image processing section 140 is configured to include a speckle pattern reduction processing section 141, an edge information calculation section 142, an image correction section 143, and an image adjustment section 144.

The speckle pattern reduction processing section 141 performs filter processing (speckle pattern reduction processing), which is for reducing a speckle pattern caused by various kinds of noise or the interference phenomenon of ultrasonic reception signals, on the ultrasonic image generated by the ultrasonic image generation section 130. In particular, in the present embodiment, the speckle pattern reduction processing section 141 reduces a speckle pattern in each pixel (pixel of interest) by setting the size of a filter according to the z-axis coordinate (coordinate in the distance direction (depth direction)) of each pixel (pixel of interest) included in the ultrasonic image and performing filter processing using the filter. For example, the speckle pattern reduction processing section 141 may set the size of the filter in the z-axis direction according to the z-axis coordinate of each pixel (pixel of interest), or may set the size of the filter in the x-axis direction according to the z-axis coordinate of each pixel (pixel of interest). For example, the speckle pattern reduction processing section 141 may set the size of the filter for a first pixel (first pixel of interest) to be equal to or greater than the size of the filter for a second pixel (second pixel of interest) whose z-axis coordinate is smaller (z-axis distance (depth) is smaller) than that of the first pixel (first pixel of interest). Details of the speckle pattern reduction processing will be described later.

The edge information calculation section 142 performs processing for calculating edge information including the strength, direction, and the like of an edge (edge information calculation processing) on each pixel (pixel of interest) whose speckle pattern has been reduced by the speckle pattern reduction processing section 141. Details of the edge information calculation processing will be described later.

The image correction section 143 performs processing for correcting the ultrasonic image generated by the ultrasonic image generation section 130 (image correction processing) based on the edge information calculated for each pixel (pixel of interest) by the edge information calculation section 142. Specifically, the image correction section 143 performs smoothing processing, which is for performing smoothing (blurring) by performing filter processing using pixel values of a plurality of other pixels, on each pixel of the ultrasonic image generated by the ultrasonic image generation section 130. The image correction section 143 performs edge sharpening processing (also referred to as edge emphasis processing), which is for sharpening (emphasizing) the edge by performing filter processing using pixel values of a plurality of other pixels, on each pixel of the ultrasonic image generated by the ultrasonic image generation section 130. The image correction section 143 performs image combining processing, which is for adding up the pixel value of the pixel subjected to the smoothing processing and the pixel value of the pixel subjected to the edge sharpening processing at a ratio corresponding to the edge information calculated by the edge information calculation section 142, on each pixel of the ultrasonic image generated by the ultrasonic image generation section 130. Details of the image correction processing (smoothing processing, edge sharpening processing, and image combining processing) will be described later.

The image adjustment section 144 performs image adjustment processing, such as processing for adjusting a gain or a dynamic range and processing for correcting each pixel value according to the depth so as to have uniform brightness in the entire image, on the ultrasonic image corrected by the image correction section 143.

The digital scan converter (DSC) 150 converts the ultrasonic image subjected to image processing by the ultrasonic image processing section 140 into a video image signal by performing supplementary processing according to the scanning line of the display unit 21, and outputs the video image signal to the display unit 21. As a result, an ultrasonic B-mode image is displayed on the display unit 21.

Based on various kinds of setting information stored in advance in the storage unit 25 or various setting signals input from the operation unit 24, the control section 160 controls each operation of the transmission and reception control section 100, the transmission pulse generation section 110, the reception processing section 120, the ultrasonic image generation section 130, the ultrasonic image processing section 140, and the digital scan converter (DSC) 150.

As described above, since the ultrasonic image processing apparatus 20 reduces a speckle pattern for the ultrasonic image generated based on the reflected wave of the ultrasonic wave and then calculates the edge information of each pixel, the outer edge of the speckle pattern is hardly recognized as an edge. Therefore, the ultrasonic image obtained by image correction processing using the edge information becomes clearer.

### 3. Ultrasonic Image Processing

### 3-1. Procedure of Ultrasonic Image Processing

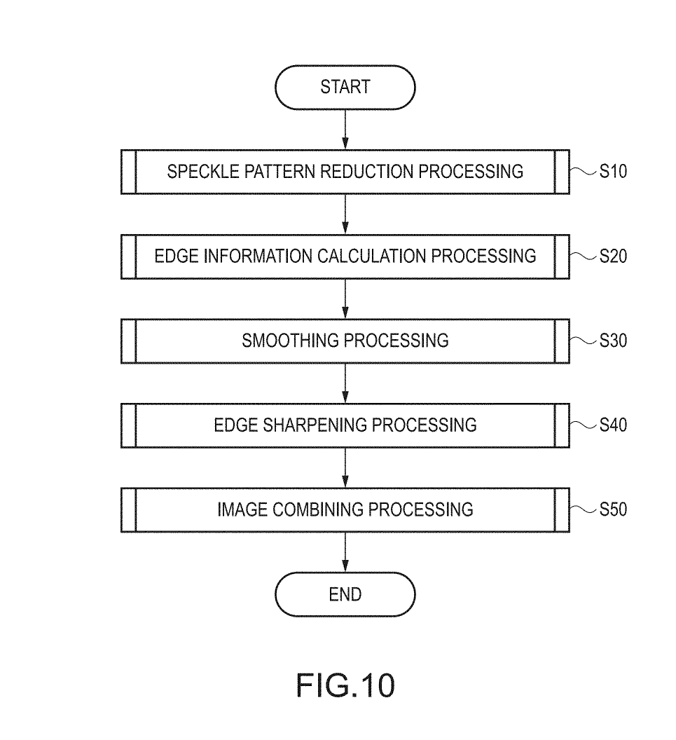

FIG. 10 is a flowchart showing the procedure of the ultrasonic image processing (ultrasonic image processing method according to the present embodiment) of the ultrasonic image processing section 140. As shown in FIG. 10, first, the speckle pattern reduction processing section 141 in the ultrasonic image processing section 140 performs speckle pattern reduction processing for reducing a speckle pattern included in the ultrasonic image generated by the ultrasonic image generation section 130 (step S10).

Then, the edge information calculation section 142 in the ultrasonic image processing section 140 performs edge information calculation processing for calculating edge information for each pixel of the ultrasonic image in which the speckle pattern has been reduced in step S10 (step S20).

Then, the image correction section 143 in the ultrasonic image processing section 140 performs smoothing processing for smoothing the ultrasonic image generated by the ultrasonic image generation section 130 (step S30).

Then, the image correction section 143 in the ultrasonic image processing section 140 performs edge sharpening processing for sharpening the edge of the ultrasonic image generated by the ultrasonic image generation section 130 (step S40).

Finally, the image correction section 143 in the ultrasonic image processing section 140 performs image combining processing for combining the ultrasonic image subjected to smoothing processing in step S30 and the ultrasonic image subjected to edge sharpening processing in step S40 based on the edge information calculated in step S20 (step S50).

In the flowchart shown in FIG. 10, the order of steps may be appropriately changed if possible. For example, the order of step S30 (smoothing processing) and step S40 (edge sharpening processing) may be interchanged.

### 3-2. Speckle Pattern Reduction Processing

The ultrasonic image has a feature that the speckle pattern is small in a shallow region (region where the z coordinate is relatively small) and the speckle pattern is large in a deep region (region where the z coordinate is relatively large). For example, in the ultrasonic image shown in FIG. 9, as the z coordinate becomes large (as the depth increases), the speckle pattern extends in the horizontal direction (x-axis direction) to become large. In the present embodiment, therefore, the speckle pattern reduction processing section 141 performs, for each pixel in a relatively shallow region, smoothing processing using the pixel values of a plurality of pixels in a relatively narrow region where the pixel is included, and performs, for each pixel in a relatively deep region, smoothing processing using the pixel values of a plurality of pixels in a relatively wide region where the pixel is included. That is, the speckle pattern reduction processing section 141 performs filter processing (speckle pattern reduction processing) for each pixel (pixel of interest) by reducing the size of the smoothing filter as the z coordinate value decreases and increasing the size of the smoothing filter as the z coordinate value increases. In this manner, the speckle pattern is effectively reduced regardless of the depth while suppressing excessive blurring of edges.

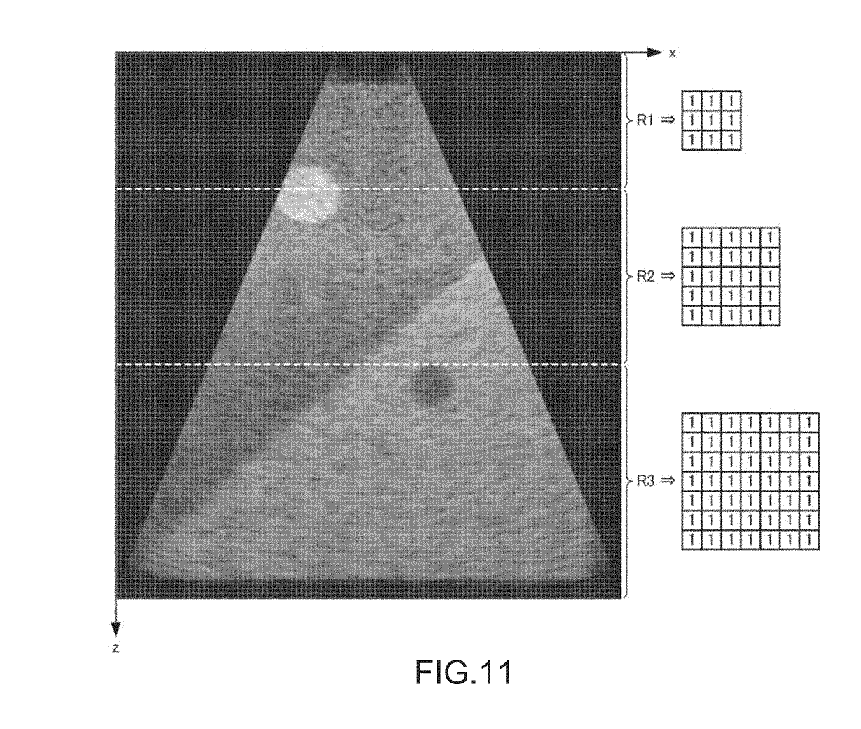

As the smoothing filter, various filters, such as a moving average filter, a Gaussian filter, and a median filter, can be applied. FIG. 11 shows an example of the relationship between each region of the ultrasonic image and the filter size to be applied in a case where a moving average filter is used as the smoothing filter. In the example shown in FIG. 11, in the ultrasonic image, a filter having a size of 3×3 (size of 9 pixels) is used in a shallowest region R1, a filter having a size of 7×7 (size of 49 pixels) is used in a deepest region R3, and a filter having a size of 5×5 (size for 25 pixels) is used in a region R2 between the shallowest region R1 and the deepest region R3. In the example shown in FIG. 11, each filter has the same size in the distance direction (z direction) and the scanning direction (x direction). However, the size in the distance direction (z direction) and the size in the scanning direction (x direction) may be different.

For example, the speckle pattern reduction processing section 141 can determine the size AF_{size} of the smoothing filter in the distance direction (z direction) with respect to the pixel of interest and the size LF_{size} of the smoothing filter in the scanning direction (x direction) with respect to the pixel of interest based on the following Expressions (1) and (2), respectively.

$$\begin{matrix}{AF}_{size} ∝ dpi \times ( A ( Mp ) \times α ( Mi ) \times \frac{n}{Freq} + B ( Mp ) \times Z + C ( Mp ) \times \frac{{Z}^{2}}{Freq} ) & ( 1 ) \\ {LF}_{size} ∝ dpi \times ( A ( Mp ) \times α ( Mi ) \times B ( Mp ) \times \frac{\vert Z - Focus \vert}{D \times Freq} + C ( Mp ) \times \frac{{Z}^{2}}{Freq} ) & ( 2 )\end{matrix}$$

In Expressions (1) and (2), Mi is an ultrasonic image generation method, and indicates by what kind of processing an ultrasonic image as the input of speckle noise reduction processing is generated. α(Mi) is a variable setting coefficient according to Mi. For example, in a case where the input ultrasonic image has already been smoothed, α(Mi) is set so that the speckle noise reduction processing becomes weak. Freq is the transmission frequency of the ultrasonic wave transmitted from the ultrasonic probe 10, and n is a transmission wave number. D is a transmission aperture diameter, and Focus is a transmission focus position (refer to FIG. 5). Z is the depth (distance) of a pixel of interest. dpi is the image resolution of the ultrasonic image. Mp is a filter processing method (for example, a filter type (a moving average filter, a Gaussian filter, a median filter, or the like)), and A(Mp), B(Mp), and C(Mp) are variable correction coefficients according to the Mp.

As shown in Expression (1), the size AF_{size} in the distance direction (z direction) is set based on the resolution of the ultrasonic wave, and is corrected in consideration of the attenuation according to the depth Z or the like. Specifically, a reference filter size is first set based on the resolution in the distance direction (term of A(Mp)×α(Mi)×n/Freq). More specifically, as the transmission frequency Freq becomes low and the transmission wave number n becomes large, the resolution in the distance direction becomes low and the speckle pattern becomes large. Therefore, the filter size is set to become large as the transmission frequency Freq becomes low and the transmission wave number n becomes large. Then, since the waveform of the ultrasonic wave becomes dull due to generation of nonlinear components according to the propagation of the ultrasonic wave, the filter size is corrected according to the depth Z of the pixel of interest (term of B(Mp)×Z). Specifically, as the pixel of interest becomes deep (depth Z increases), the waveform becomes dull and the speckle pattern becomes large. Therefore, the filter size is corrected so as to become large as the pixel of interest becomes deep (depth Z increases). In addition, since the ultrasonic wave attenuates (has a lower frequency) according to its propagation, the filter size is corrected by the square of the depth Z of the pixel of interest (term of C(Mp)×Z²/Freq). Specifically, as the transmission frequency Freq becomes low and the pixel of interest becomes deep (depth Z increases), the ultrasonic wave attenuates (has a lower frequency) to increase a speckle pattern. Therefore, the filter size is corrected so as to become large as the transmission frequency Freq becomes low and the pixel of interest becomes deep (depth Z increases). Then, the filter size is changed to a size suitable for the ultrasonic image using the image resolution dpi.

As shown in Expression (2), the size LF_{size} in the scanning direction (x direction) is set based on the ultrasonic image generation method, and is corrected in consideration of attenuation and the resolution (beam width) of the ultrasonic wave according to the depth Z. Specifically, a reference filter size is first set using the ultrasonic image generation method Mi (term of A(Mp)×α(Mi)). Then, since the beam width of the ultrasonic wave increases due to deviation from the transmission focus position Focus, the filter size is corrected according to the depth Z of the pixel of interest (term of B(Mp)×|Z−Focus|/(D×Freq)). Specifically, as the deviation in the distance direction (depth direction) from the transmission focus position Focus increases and the transmission aperture diameter D or the transmission frequency Freq decreases, the beam width of the ultrasonic wave increases and the speckle pattern increases. Therefore, the filter size is corrected so as to become large as the deviation in the distance direction (depth direction) from the transmission focus position Focus increases and the transmission aperture diameter D or the transmission frequency Freq decreases. In addition, since the ultrasonic wave attenuates (has a lower frequency) according to its propagation, the filter size is corrected by the square of the depth Z (term of C(Mp)×Z²/Freq). Specifically, as the transmission frequency Freq becomes low and the pixel of interest becomes deep (depth Z increases), the ultrasonic wave attenuates (has a lower frequency) to increase a speckle pattern. Therefore, the filter size is corrected so as to become large as the transmission frequency Freq becomes low and the pixel of interest becomes deep (depth Z increases). Then, the filter size is changed to a size suitable for the ultrasonic image using the image resolution dpi.

In Expressions (1) and (2), the coefficient α(Mi), the transmission frequency Freq, the transmission wave number n, the transmission aperture diameter D, the transmission focus position Focus, the image resolution dpi, and the correction coefficients A(Mp), B(Mp), and C(Mp) are set before the speckle pattern reduction processing is performed. Therefore, the size AF_{size} in the distance direction (z direction) and the size LF_{size} in the scanning direction (x direction) depend on the depth (distance) Z of the pixel of interest, and increase as Z increases.

In practice, the filter size is an integral value. Accordingly, values obtained by rounding off AF_{size} and LF_{size} to integral values are determined as the size in the distance direction (z direction) and the size in the scanning direction (x direction).

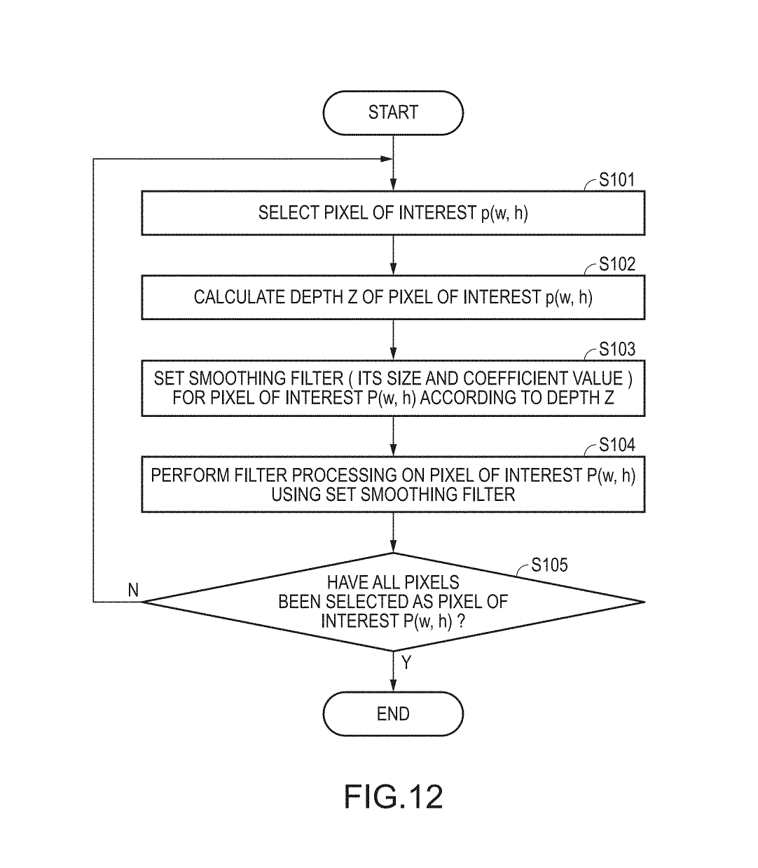

FIG. 12 is a flowchart showing an example of the procedure of the speckle pattern reduction processing (processing of step S10 in FIG. 10) of the speckle pattern reduction processing section 141.

In the example shown in FIG. 12, the speckle pattern reduction processing section 141 selects a pixel of interest p(w, h) first (step S101). For example, in step S101, a pixel of interest p(0, 0) (pixel whose both x and z coordinates are 0) is selected.

Then, the speckle pattern reduction processing section 141 calculates the depth Z of the pixel of interest p(w, h) selected in step S101 (step S102). The depth Z of the pixel of interest p(w, h) may be a z-coordinate value itself, or may be calculated by multiplying the z-coordinate value by a predetermined coefficient.

Then, the speckle pattern reduction processing section 141 sets a smoothing filter (its size and coefficient value) for the pixel of interest p(w, h) according to the depth Z calculated in step S102 (step S103). The size of the smoothing filter is set using Expressions (1) and (2), for example. The coefficient value of the smoothing filter is set based on a filter processing method Mp (filter type (a moving average filter, a Gaussian filter, or the like)) set in advance. In a case where the median filter is set as a smoothing filter, there is no concept of the coefficient value. In this case, therefore, the speckle pattern reduction processing section 141 may set only the size of the smoothing filter, or may set the size and the number of iterations.

Then, the speckle pattern reduction processing section 141 performs filter processing on the pixel of interest p(w, h) using the smoothing filter set in step S103 (step S104). By the filter processing, the speckle pattern in the pixel of interest p(w, h) is reduced.

Then, the speckle pattern reduction processing section 141 determines whether or not all the pixels of the ultrasonic image have been selected as the pixel of interest p(w, h) (step S105). In a case where there is an unselected pixel (N in step S105), the speckle pattern reduction processing section 141 selects the next pixel of interest p(w, h) (step S101), and performs the processing from step S102 again. In a case where there is no unselected pixel (Y in step S105), the speckle pattern reduction processing is ended.

### 3-3. Edge Information Calculation Processing

In the present embodiment, the edge information calculation section 142 can perform, for example, Prewitt processing as processing for calculating edge information (strength and direction of an edge) for each pixel of the ultrasonic image subjected to the speckle pattern reduction processing. In the Prewitt processing, the edge strength Δfₓ in the scanning direction (x direction) and the edge strength Δf_{z} in the distance direction (z direction) with respect to the pixel of interest p(w, h) of the ultrasonic image are calculated by the following Equations (3) and (4), respectively.

$$\begin{matrix}Δ {f}_{x} = \frac{1}{{M}_{z}} ( {\sum }_{j = - {M}_{z}}^{{M}_{z}} {P}_{w + {M}_{x} h + j} - {\sum }_{j = - {M}_{z}}^{{M}_{z}} {P}_{w - {M}_{x} , h + j} ) & ( 3 ) \\ Δ {f}_{z} = \frac{1}{{M}_{x}} ( {\sum }_{j = - {M}_{x}}^{{M}_{x}} {P}_{w + i , h + {M}_{z}} - {\sum }_{j = - {M}_{x}}^{{M}_{x}} {P}_{w + i , h - {M}_{z}} ) & ( 4 )\end{matrix}$$

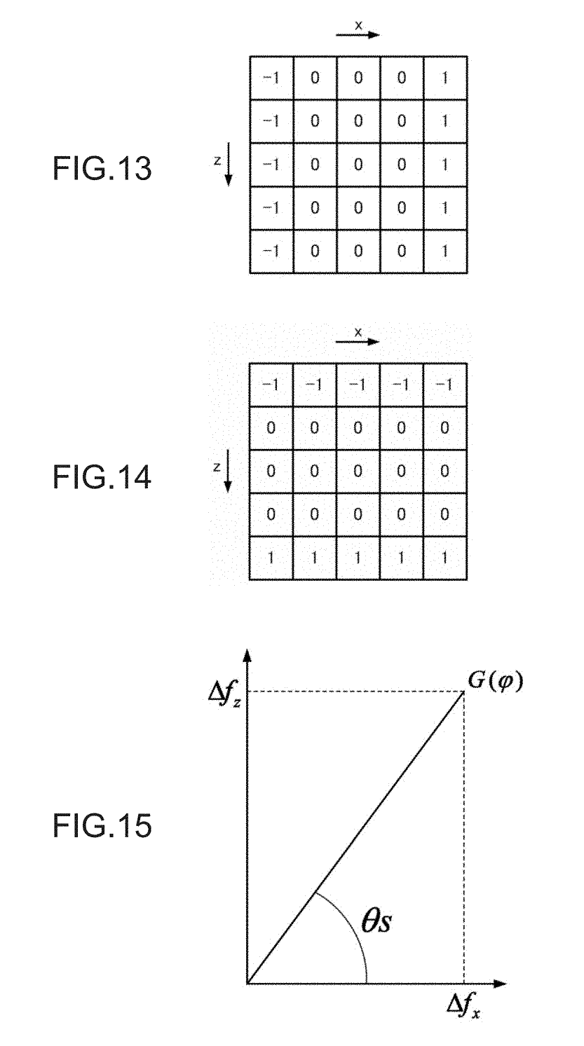

In Equation (3), P is a pixel value, and the edge strength Δfₓ in the scanning direction (x direction) is calculated from the pixel values P of “(2Mₓ+1)×(2M_{z}+1)” pixels centered on the pixel of interest p(w, h). The edge strength Δfₓ in the scanning direction (x direction) can be calculated by filter processing using “(2Mₓ+1)×(2M_{z}+1)” pixels centered on the pixel of interest p(w, h), in which a desired filter having a size of (2Mₓ+1)×(2M_{z}+1) is used. FIG. 13 shows an example of a 5×5 filter that is used for calculation of the edge strength Δfₓ in the scanning direction (x direction) in the case of Mₓ=M_{z}=2.

Similarly, in Equation (4), P is a pixel value, and the edge strength Δf_{z} in the distance direction (z direction) is calculated from the pixel values P of “(2Mₓ+1)×(2M_{z}+1)” pixels centered on the pixel of interest p (w, h). The edge strength Δf_{z} in the distance direction (z direction) can be calculated by filter processing using “(2Mₓ+1)×(2M_{z}+1)” pixels centered on the pixel of interest p(w, h), in which a desired filter having a size of (2Mₓ+1)×(2M_{z}+1) is used. FIG. 14 shows an example of a 5×5 filter that is used for calculation of the edge strength Δf_{z} in the distance direction (z direction) in the case of Mₓ=M_{z}=2.

From the edge strength Δfₓ in the scanning direction (x direction) calculated by Equation (3) and the edge strength Δf_{z} in the distance direction (z direction) calculated by Equation (4), an edge strength G(ϕ) for the pixel of interest p(w, h) is calculated by the following Equation (5).

G(φ)=\sqrt{Δfₓ²+Δf_{z}²} (5)

An edge direction θₛ for the pixel of interest p(w, h) is calculated by the following Equation (6).

$${θ}_{s} = {tan}^{- 1} ( \frac{Δ {f}_{z}}{Δ {f}_{x}} ) \qquad ( 6 )$$

FIG. 15 shows the relationship among the edge strength G(ϕ) and the edge direction θₛ for the pixel of interest p(w, h), the edge strength Δfₓ in the scanning direction (x direction), and the edge strength Δf_{z} in the distance direction (z direction).

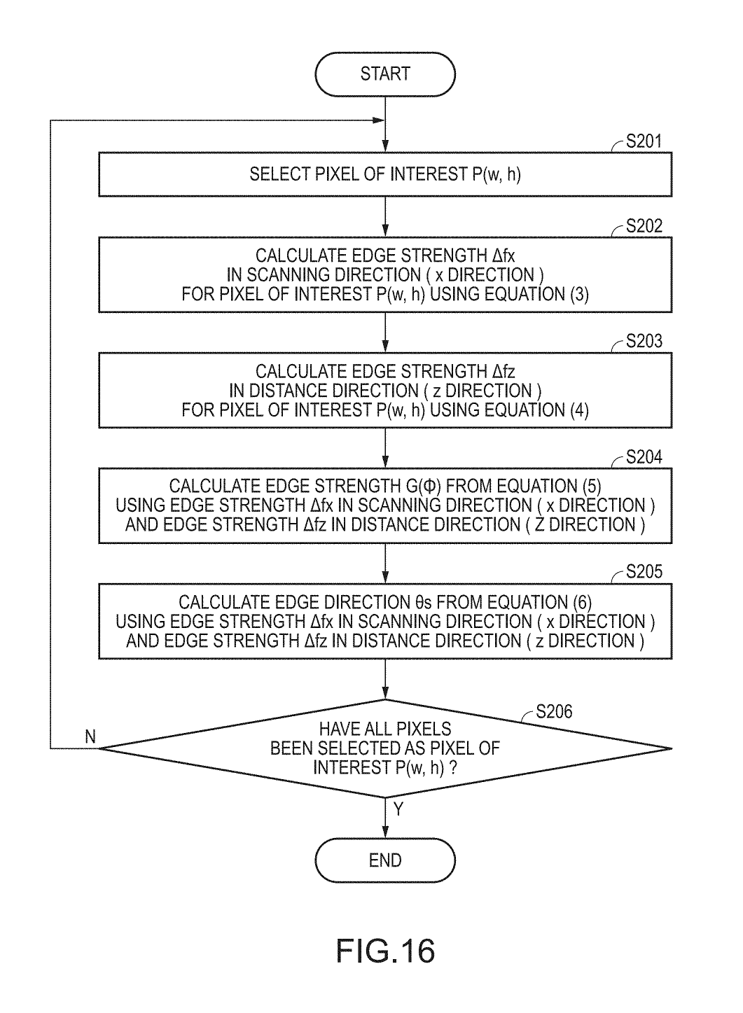

FIG. 16 is a flowchart showing an example of the procedure of the edge information calculation processing (processing of step S20 in FIG. 10) of the edge information calculation section 142.

In the example shown in FIG. 16, the edge information calculation section 142 selects the pixel of interest p(w, h) first (step S201). For example, in step S201, a pixel of interest p(0, 0) (pixel whose both x and z coordinates are 0) is selected.

Then, the edge information calculation section 142 calculates the edge strength Δfₓ in the scanning direction (x direction) for the pixel of interest p(w, h) selected in step S201 using Equation (3) (step S202).

Then, the edge information calculation section 142 calculates the edge strength Δf_{z} in the distance direction (z direction) for the pixel of interest p(w, h) selected in step S201 using Equation (4) (step S203).

Then, the edge information calculation section 142 calculates the edge strength G(ϕ) from Equation (5) using the edge strength Δfₓ in the scanning direction (x direction) calculated in step S202 and the edge strength Δf_{z} in the distance direction (z direction) calculated in step S203 (step S204).

Then, the edge information calculation section 142 calculates the edge direction θₛ from Equation (6) using the edge strength Δfₓ in the scanning direction (x direction) calculated in step S202 and the edge strength Δf_{z} in the distance direction (z direction) calculated in step S203 (step S205).

Then, the edge information calculation section 142 determines whether or not all the pixels of the ultrasonic image have been selected as the pixel of interest p (w, h) (step S206). In a case where there is an unselected pixel (N in step S206), the edge information calculation section 142 selects the next pixel of interest p(w, h) (step S201), and performs the processing from step S202 again. In a case where there is no unselected pixel (Y in step S206), the edge information calculation processing is ended.

### 3-4. Smoothing Processing

In the present embodiment, as the smoothing processing on the ultrasonic image (original ultrasonic image) generated by the ultrasonic image generation section 130, the image correction section 143 can perform filter processing using a smoothing filter, such as a moving average filter, a Gaussian filter, and a median filter.

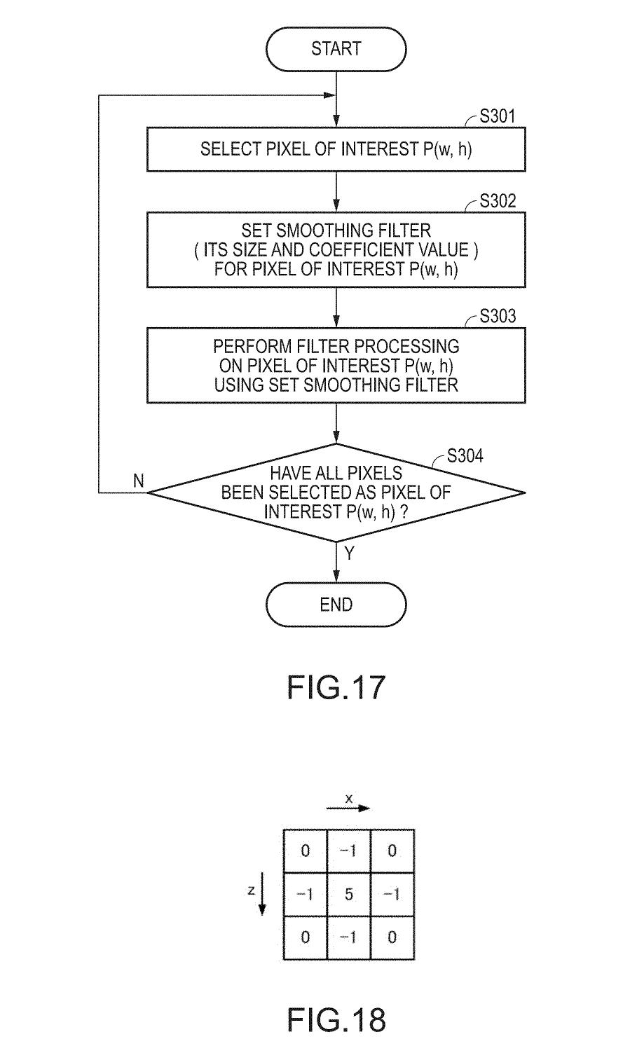

FIG. 17 is a flowchart showing an example of the procedure of the smoothing processing (processing of step S30 in FIG. 10) of the image correction section 143.

In the example shown in FIG. 17, the image correction section 143 selects the pixel of interest p(w, h) first (step S301). For example, in step S301, a pixel of interest p(0, 0) (pixel whose both x and z coordinates are 0) is selected.

Then, the image correction section 143 sets a smoothing filter (its size and coefficient value) for the pixel of interest p(w, h) selected in step S301 (step S302). In step S302, the image correction section 143 may set a smoothing filter for performing strong smoothing in the edge direction θₛ for the pixel of interest p(w, h).

Then, the image correction section 143 performs filter processing on the pixel of interest p(w, h) using the smoothing filter set in step S302 (step S303).

Then, the image correction section 143 determines whether or not all the pixels of the ultrasonic image have been selected as the pixel of interest p(w, h) (step S304). In a case where there is an unselected pixel (N in step S304), the image correction section 143 selects the next pixel of interest p (w, h) (step S301), and performs the processing from step S302 again. In a case where there is no unselected pixel (Y in step S304), the smoothing processing is ended.

### 3-5. Edge Sharpening Processing

In the present embodiment, as the edge sharpening processing on the ultrasonic image (original ultrasonic image) generated by the ultrasonic image generation section 130, the image correction section 143 can perform filter processing using an edge sharpening filter that increases a pixel value for pixels in an edge portion and reduces a pixel value for pixels in a region (flat region) including no edge. FIG. 18 shows an example of a 3×3 filter used for edge sharpening processing.

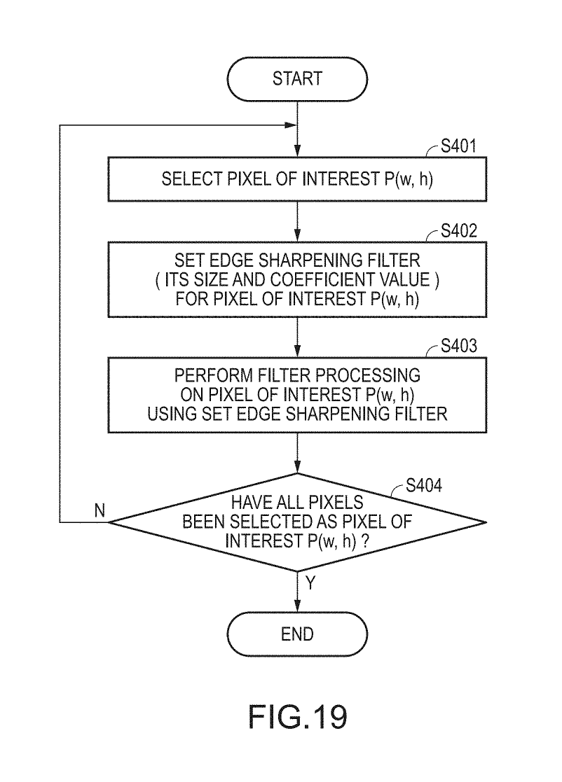

FIG. 19 is a flowchart showing an example of the procedure of the edge sharpening processing (processing of step S40 in FIG. 10) of the image correction section 143.

In the example shown in FIG. 19, the image correction section 143 selects the pixel of interest p(w, h) first (step S401). For example, in step S401, a pixel of interest p(0, 0) (pixel whose both x and z coordinates are 0) is selected.

Then, the image correction section 143 sets an edge sharpening filter (its size and coefficient value) for the pixel of interest p(w, h) selected in step S401 (step S402). In step S402, the image correction section 143 may set an edge sharpening filter for performing strong sharpening in a direction perpendicular to the edge direction θₛ for the pixel of interest p(w, h).

Then, the image correction section 143 performs filter processing on the pixel of interest p(w, h) using the edge sharpening filter set in step S402 (step S403).

Then, the image correction section 143 determines whether or not all the pixels of the ultrasonic image have been selected as the pixel of interest p(w, h) (step S404). In a case where there is an unselected pixel (N in step S404), the image correction section 143 selects the next pixel of interest p(w, h) (step S401), and performs the processing from step S402 again. In a case where there is no unselected pixel (Y in step S404), the edge sharpening processing is ended.

### 3-6. Image Combining Processing

In the present embodiment, as the image combining processing, the image correction section 143 can perform processing for combining the pixel value after the smoothing processing (processing of step S30 in FIG. 10) and the pixel value after the edge sharpening processing (processing of step S40 in FIG. 10) according to the edge strength G(ϕ) with the pixel value of each pixel (pixel of interest p(w, h)) of the ultrasonic image (original ultrasonic image) generated by the ultrasonic image generation section 130. The pixel value Pc_{w}, h of the pixel of interest p(w, h) after the combining is calculated by the following Equation (7), for example.

Pc_{w,h}=P_{w,h}+{I×(1.0−G(φ)′)×(Pc_{w,h}−P_{w,h})}+{J×G(φ)′×(Pc_{w,h}−P_{w,h})} (7)

In Equation (7), P_{w, h} is the pixel value of the pixel of interest p(w, h) in the ultrasonic image (original ultrasonic image) generated by the ultrasonic image generation section 130. Pr_{w, h} is the pixel value of the pixel of interest p(w, h) subjected to the smoothing processing (processing of step S30 in FIG. 10), and Pe_{w, h} is the pixel value of the pixel of interest p (w, h) subjected to the edge sharpening processing (processing of step S40 in FIG. 10). G(ϕ)′ is obtained by normalizing the edge strength G(ϕ) for the pixel of interest p(w, h) to a value in the range of 0 to 1.0. I is a coefficient having a value in the range of 0 to 1.0 indicating the strength of smoothing processing, and J is a coefficient having a value in the range of 0 to 1.0 indicating the strength of edge sharpening processing. The coefficients I and J are set by the user through the user interface screen displayed on the display unit 21 of the ultrasonic image processing apparatus 20, for example.

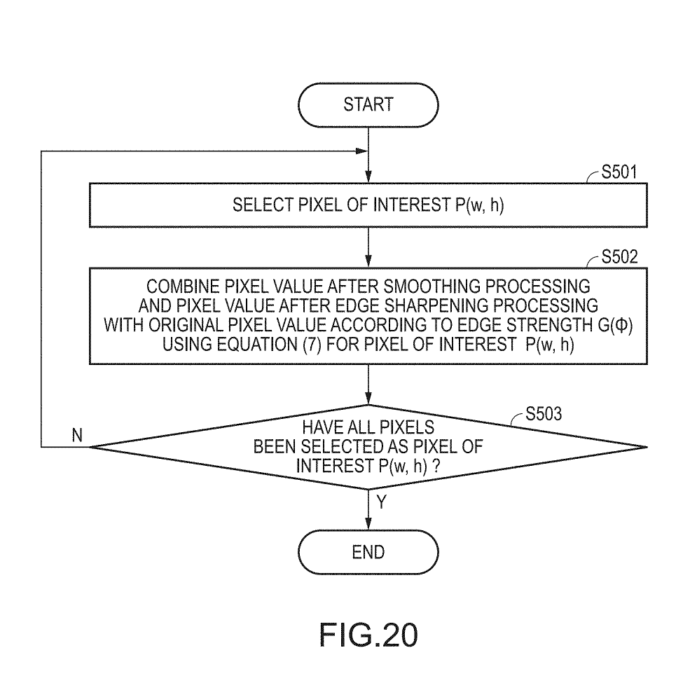

FIG. 20 is a flowchart showing an example of the procedure of the image combining processing (processing of step S50 in FIG. 10) of the image correction section 143.

In the example shown in FIG. 20, the image correction section 143 selects the pixel of interest p(w, h) first (step S501). For example, in step S501, a pixel of interest p(0, 0) (pixel whose both x and z coordinates are 0) is selected.

Then, for the pixel of interest p(w, h) selected in step S501, the image correction section 143 combines the pixel value after the smoothing processing and the pixel value after the edge sharpening processing with the original pixel value according to the edge strength G(ϕ) using Equation (7) (step S502).

Then, the image correction section 143 determines whether or not all the pixels of the ultrasonic image have been selected as the pixel of interest p(w, h) (step S503). In a case where there is an unselected pixel (N in step S503), the image correction section 143 selects the next pixel of interest p(w, h) (step S501), and performs the processing from step S502 again. In a case where there is no unselected pixel (Y in step S503), the image combining processing is ended.

### 4. Function and Effect of Ultrasonic Image Apparatus (Ultrasonic Image Processing Apparatus)

The ultrasonic image apparatus 1 (ultrasonic image processing apparatus 20) according to the present embodiment generates an ultrasonic image based on the reflected wave of the ultrasonic wave transmitted to the object. Since nonlinear components are generated according to the propagation of the ultrasonic wave, the waveform of the ultrasonic wave becomes dull and the ultrasonic wave attenuates (has a lower frequency). As a result, in the generated ultrasonic image, the speckle pattern increases as the coordinate on the z axis corresponding to the distance direction (depth direction) in which the ultrasonic wave propagates becomes large. Therefore, in the speckle reduction processing, the ultrasonic image apparatus 1 (ultrasonic image processing apparatus 20) according to the present embodiment sets the size of the smoothing filter in the z-axis direction and the size of the smoothing filter in the x-axis direction to be large for a pixel having a large z coordinate (having a large distance (depth) in which the ultrasonic wave propagates), thereby performing smoothing using the pixel values of a larger number of pixels. That is, according to the ultrasonic image apparatus 1 (ultrasonic image processing apparatus 20) of the present embodiment, by performing smoothing processing using a filter having an appropriate size corresponding to the size of the speckle pattern for each pixel, it is possible to effectively reduce the speckle pattern and to suppress blurring of edges due to smoothing as much as possible. According to the ultrasonic image apparatus 1 (ultrasonic image processing apparatus 20) of the present embodiment, since it is difficult for the outer edge of the speckle pattern to be recognized as an edge in the subsequent edge information calculation processing, it is possible to accurately calculate the edge information (strength or direction of the edge) for each pixel. Therefore, according to the ultrasonic image apparatus 1 (ultrasonic image processing apparatus 20) of the present embodiment, by performing image correction processing using the highly accurate edge information calculated for each pixel, it is possible to generate and display a clearer ultrasonic image in which the sharpness of the edge region is emphasized.

### 5. Modification Examples

The invention is not limited to the present embodiment, and can be modified within the scope of the invention.

For example, in the embodiment described above, the ultrasonic transducer element 12 has a configuration using a piezoelectric element, but the invention is not limited thereto. For example, a capacitive element, such as a capacitive micro-machined ultrasonic transducer (c-MUT), may be used, or a bulk type element may be used.

For example, in the embodiment described above, in the ultrasonic transducer device 11, a plurality of ultrasonic transducer elements 12 are arranged in a matrix (refer to FIG. 2), but the invention is not limited thereto. For example, the ultrasonic transducer elements 12 in adjacent two columns may be arranged alternately (in a so-called zigzag manner).

For example, in the embodiment described above, the size of the filter in the distance direction (depth direction) and the size of the filter in the scanning direction (azimuth direction) used for the speckle pattern reduction processing are separately calculated based on Expression (1) and Expression (2), but the invention is not limited thereto. For example, the size of the filter in the distance direction (depth direction) and the size of the filter in the scanning direction (azimuth direction) may be calculated so as to be always the same based on either one of Expression (1) and Expression (2).

In addition, for example, although the speckle noise reduction processing of the speckle pattern reduction processing section 141 and the edge information calculation processing of the edge information calculation section 142 are performed after the processing of the logarithmic transformation section 134 in the embodiment described above, the speckle noise reduction processing of the speckle pattern reduction processing section 141 and the edge information calculation processing of the edge information calculation section 142 may be performed after the processing of the detection processing section 133. The speckle noise reduction processing of the speckle pattern reduction processing section 141, the edge information calculation processing of the edge information calculation section 142, and the image correction processing of the image correction section 143 may be performed after the image adjustment processing of the image adjustment section 144, or may be performed after the processing of the digital scan converter (DSC) 150.

For example, although the ultrasonic image processing apparatus 20 has been described as an example of the ultrasonic image processing apparatus according to the invention in the above embodiment, the ultrasonic image processing apparatus according to the invention may be configured to include the ultrasonic probe 10 and the ultrasonic image processing apparatus 20.

For example, although the ultrasonic image apparatus 1 (ultrasonic image processing apparatus 20) generates, processes, and displays a two-dimensional ultrasonic image in the embodiment described above, the ultrasonic image apparatus 1 (ultrasonic image processing apparatus 20) may generate, process, and display a three-dimensional ultrasonic image.

The embodiments and the modification examples described above are just examples, and the invention is not limited to these. For example, each embodiment and each modification example can be appropriately combined.

The invention includes substantially the same configuration (for example, a configuration with the same function, method, and result or a configuration with the same object and effect) as the configuration described in each embodiment. The invention includes a configuration in which a non-essential portion of the configuration described in the embodiment is replaced. The invention includes a configuration capable of achieving the same effect as in the configuration described in each embodiment or a configuration capable of achieving the same object. The invention includes a configuration obtained by adding a known technique to the configuration described in the embodiment.

The entire disclosure of Japanese Patent Application No. 2017-034466 filed Feb. 27, 2017 is expressly incorporated by reference herein.

What is claimed is:

1. An ultrasonic image processing apparatus for processing an ultrasonic image including a plurality of pixels arranged in a direction of a first axis corresponding to a scanning direction of an ultrasonic wave transmitted to an object and a direction of a second axis corresponding to a distance direction in which the ultrasonic wave propagates, each of the plurality of pixels having coordinates based on a reflection position of the ultrasonic wave and a pixel value based on a strength of a reflected wave of the ultrasonic wave, the apparatus comprising:
a speckle pattern reduction processing section that sets a size of a filter according to a coordinate on the second axis of a pixel of interest included in the ultrasonic image and performs filter processing using the filter to reduce a speckle pattern in the pixel of interest; and
an edge information calculation section that calculates edge information for the pixel of interest in which the speckle pattern has been reduced.

2. The ultrasonic image processing apparatus according to claim 1,
wherein the speckle pattern reduction processing section sets the size of the filter in the second axis direction according to the coordinate of the pixel of interest on the second axis.

3. The ultrasonic image processing apparatus according to claim 1,
wherein the speckle pattern reduction processing section sets the size of the filter in the first axis direction according to the coordinate of the pixel of interest on the second axis.

4. The ultrasonic image processing apparatus according to claim 1,
wherein the speckle pattern reduction processing section sets the size of the filter for a first pixel of interest to be equal to or greater than the size of the filter for a second pixel of interest whose coordinate on the second axis is smaller than that of the first pixel of interest.

5. The ultrasonic image processing apparatus according to claim 1, further comprising:
an ultrasonic image generation section that generates the ultrasonic image based on a reflected wave of the ultrasonic wave transmitted to the object.

6. The ultrasonic image processing apparatus according to claim 1, further comprising:
an image correction section that corrects the ultrasonic image based on the edge information.

7. An ultrasonic image processing method for processing an ultrasonic image including a plurality of pixels arranged in a direction of a first axis corresponding to a scanning direction of an ultrasonic wave transmitted to an object and a direction of a second axis corresponding to a distance direction in which the ultrasonic wave propagates, each of the plurality of pixels having coordinates based on a reflection position of the ultrasonic wave and a pixel value based on a strength of a reflected wave of the ultrasonic wave, the method comprising:
setting a size of a filter according to a coordinate on the second axis of a pixel of interest included in the ultrasonic image;
performing filter processing using the filter to reduce a speckle pattern in the pixel of interest; and
calculating edge information for the pixel of interest in which the speckle pattern has been reduced.
